(19) United States
(12) Patent Application Publication
(10) Pub. No.: US 2019/0133526 A1
(43) Pub. Date: May 9, 2019

(54) WEARABLE APPARATUS AND METHOD OF CONTROLLING THE SAME

(71) Applicant: BOE Technology Group Co., Ltd., Beijing (CN)

(72) Inventors: Xin Li, Beijing (CN); Honglei Zhang, Beijing (CN)

(21) Appl. No.: 15/919,290

(22) Filed: Mar. 13, 2018

(30) Foreign Application Priority Data
Nov. 9, 2017 (CN) 201711097989.2

(51) Int. Cl. A61B 5/00; A61B 5/145; G06F 21/32

(52) U.S. Cl. CPC A61B 5/6826; A61B 5/145; A61B 5/7405; A61B 5/742; G06F 21/32

(57) ABSTRACT

The present disclosure provides a wearable apparatus and a method of controlling the wearable apparatus. The wearable apparatus includes a body and an identity characteristic extraction device, a biometric characteristic parameter detector and a processor that are disposed onto the body. The identity characteristic extraction device is electrically connected with the processor and configured to acquire identity characteristic information of a user of the wearable apparatus. The biometric characteristic parameter detector is electrically connected with the processor and configured to detect a real-time biometric characteristic parameter of the user. The processor is configured to, according to the acquired identity characteristic information, control the biometric characteristic parameter detector to detect the real-time biometric characteristic parameter of the user and compare the detected real-time biometric characteristic parameter with a reference biometric characteristic parameter so as to determine whether the real-time biometric characteristic parameter is abnormal or not.

### CROSS-REFERENCE TO RELATED APPLICATION

This application claims the priority benefit of the Chinese Patent Application No. 201711097989.2 filed on Nov. 9, 2017 in the State Intellectual Property Office of China, the whole disclosure of which is incorporated herein by reference.

### TECHNICAL FIELD

Embodiments of the present disclosure generally relate to technical field of detection, and particularly to a wearable apparatus and a method of controlling a wearable apparatus.

### DESCRIPTION OF THE RELATED ART

With high-speed development of the society economy and technology, people pay more attention on health matter. Currently, most health examination s need blood examination.

Conventional blood examination apparatuses have a complex structure and a higher cost, and usually include a blood cell analyzer, a biochemical analyzer, an enzyme labeling device, a spectrophotometer, a hematimeter, etc. If a user wants a blood examination, he/she has to go to a specific health examination central or hospital and the blood examination process is complex.

### SUMMARY

In order to eliminate at least one of the above and other problems and defects in prior arts, the present disclosure is made.

In an aspect, an embodiment of the present disclosure provides a wearable apparatus, comprising a body and an identity characteristic extraction device, a biometric characteristic parameter detector and a processor that are disposed onto the body;

the identity characteristic extraction device is electrically connected with the processor and is configured to acquire identity characteristic information of a user of the wearable apparatus;

the biometric characteristic parameter detector is electrically connected with the processor and is configured to detect a real-time biometric characteristic parameter of the user; and

the processor is configured to, according to the acquired identity characteristic information, control the biometric characteristic parameter detector to detect the real-time biometric characteristic parameter of the user and compare the detected real-time biometric characteristic parameter with a reference biometric characteristic parameter so as to determine whether the detected real-time biometric characteristic parameter is abnormal or not.

In one embodiment, the processor is configured to compare the acquired identity characteristic information of the user with the reference identity characteristic information, and, in response to matching between the acquired identity characteristic information of the user and the reference identity characteristic information, control the biometric characteristic parameter detector to detect the real-time biometric characteristic parameter of the user. In one embodiment, the processor is further configured to, in response to determining the detected real-time biometric characteristic parameter is abnormal, analyze the detected real-time biometric characteristic parameter and output health information.

In one embodiment, the health information comprises at least one of a potential disease of the user and recommendation for health improvement for the user.

In one embodiment, the identity characteristic information includes fingerprint information of the user, and the real-time biometric characteristic parameter includes at least one of a blood parameter, a blood sugar parameter and a blood pressure parameter of the user.

In one embodiment, the wearable apparatus further comprises a display device electrically connected with the processor and configured to display the health information.

In one embodiment, the wearable apparatus further comprises a touch device electrically connected with the processor and configured to, in response to mismatching between the acquired identity characteristic information and the reference identity characteristic information, receive identity authentication information entered by the user, and the processor is further configured to, according to the received identity authentication information, control the biometric characteristic parameter detector to detect the real-time biometric characteristic parameter of the user.

In one embodiment, the wearable apparatus further comprises a distance detection device electrically connected with the processor and configured to detect a distance between the wearable apparatus and at least one electronic apparatus; and the processor is further configured to, according to the detected distance, determine whether or not the wearable apparatus mates with the at least one electronic apparatus and control the at least one electronic apparatus to perform at least one operation in response to success mating of the wearable apparatus with the at least one electronic apparatus.

In one embodiment, the wearable apparatus further comprises an air detection device electrically connected with the processor and configured to detect air quality in an environment where the wearable apparatus is located, and the processor is further configured to feed back the air quality to the user.

In one embodiment, the wearable apparatus is a glove, and the identity characteristic extraction device is disposed at a fingertip portion of the glove and configured to acquire the user's fingerprint information in a state where the user wears the glove.

In one embodiment, the biometric characteristic parameter detector comprises a non-invasive medical examination device.

In one embodiment, the wearable apparatus further comprises an audio player electrically connected with the processor and configured to play in a form of voice the health information outputted by the processor.

In another aspect, an embodiment of the present disclosure provides a method of controlling a wearable apparatus, the method comprising:

acquiring identity characteristic information of a user of the wearable apparatus;

determining whether or not a real-time biometric characteristic parameter of the user is to be detected according to the acquired identity characteristic information of the user; and

comparing, after detecting the real-time biometric characteristic parameter of the user, the detected real-time biometric characteristic parameter with a reference biometric characteristic parameter so as to determine whether the detected real-time biometric characteristic parameter is abnormal or not.

In one embodiment, the determining whether or not a real-time biometric characteristic parameter of the user is to be detected according to the identity characteristic information of the user comprises:

comparing the acquired identity characteristic information of the user with the reference identity characteristic information, and, in response to matching between the acquired identity characteristic information of the user and the reference identity characteristic information, detecting the real-time biometric characteristic parameter of the user.

In one embodiment, the method further comprises analyzing the detected real-time biometric characteristic parameter and outputting health information when determining the detected real-time biometric characteristic parameter is abnormal.

In one embodiment, the health information comprises at least one of a potential disease of the user and recommendation for health improvement for the user.

In one embodiment, the identity characteristic information includes fingerprint information of the user, and the real-time biometric characteristic parameter includes at least one of a blood parameter, a blood sugar parameter and a blood pressure parameter.

In one embodiment, the method further comprises: receiving identity authentication information entered by the user to perform identity authentication for the user when the identity characteristic information does not match the reference identity characteristic information, and, according to identity authentication result, determining whether or not the real-time biometric characteristic parameter of the user is to be detected.

In one embodiment, the method further comprises:

detecting a distance between the wearable apparatus and at least one electronic apparatus;

determining whether or not the wearable apparatus mates with the at least one electronic apparatus according to the detected distance; and

controlling the at least one electronic apparatus to perform at least one operation when the wearable apparatus successfully mates with the at least one electronic apparatus.

In one embodiment, the determining whether or not the wearable apparatus mates with the at least one electronic apparatus according to the detected distance comprises:

comparing the detected distance with a distance threshold, and controlling the wearable apparatus to mate with the at least one electronic apparatus when the detected distance is less than the distance threshold.

### BRIEF DESCRIPTION OF THE DRAWINGS

FIG. 1 is a structural schematic view of a wearable apparatus according to an embodiment of the present disclosure; and

FIG. 2 is a flow chart of a method of controlling a wearable apparatus according to an embodiment of the present disclosure.

### DETAILED DESCRIPTION OF PREFERRED EMBODIMENTS

In order to provide a more clear understanding of objects, technique features and advantages of the present disclosure, the disclosure will be further described hereinafter in detail in conjunction with exemplary embodiments and with reference to the attached drawings.

In the description of the present disclosure, unless otherwise defined, “a plurality of” or “more” means two or more, terms such as “up”, “down”, “left”, “right”, “inside”, “outside” and the like are directed to orientations or positions based on orientations or positions illustrated in the drawings and are merely used for simply and conveniently describing the present disclosure, instead of indicating or implying that the indicated element or component has to be at a specific orientation or position, and be configured and operated at a specific orientation or position, and thus should not be understood to limit the present disclosure.

In the description of the disclosure, it is noted that unless otherwise defined or specified, terms such as “mount”, “connect”, “couple” or the like should be understood broadly, for example, may be directed to a fixed connection, or may be a removable connection, or may be a integral connection; may be understood as a mechanical connection, or an electrical connection; may be understood as a direct connection, or may be an indirect connection by a medium. Those skilled in the art may understand specific meanings of the above terms in the present disclosure according to actual situations.

A further detail description of exemplary embodiments of the present disclosure will be made in conjunction with the drawings and examples. The following embodiments are intended to describe the present disclosure, instead of limiting the present disclosure.

Embodiments of the present disclosure provide a wearable apparatus including a body, such as, a body suitable to be worn by a user, and an identity characteristic extraction device, a biometric characteristic parameter detector and a processor that are disposed onto the body. The processor is electrically connected with the identity characteristic extraction device and the biometric characteristic parameter detector respectively.

The identity characteristic extraction device is configured to acquire identity characteristic information of the user of the wearable apparatus.

The biometric characteristic parameter detector is configured to detect a real-time biometric characteristic parameter of the user.

The processor may include a microprocessor and is configured to, according to the identity characteristic information acquired by the identity characteristic extraction device, control the biometric characteristic parameter detector to detect the real-time biometric characteristic parameter of the user, and compare the detected real-time biometric characteristic parameter with a reference biometric characteristic parameter so as to determine whether the real-time biometric characteristic parameter is abnormal or not.

The wearable apparatus according to embodiments of the present disclosure may be of various types such as a glove, glasses, or the like.

The identity characteristic extraction device is used for acquiring identity characteristic information of the user of the wearable apparatus, and the identity characteristic information is a unique identification of the user. The identity characteristic information may include various types, such as fingerprint information, pupil information, or the like. Based on the type of the acquired identity characteristic information, the identity characteristic extraction device may include various types, such as a fingerprint identification sensor configured to acquire fingerprint information, an image identification sensor configured to acquire the pupil information, or the like. The specific form of the identity characteristic extraction device may be selected and set according to actual situation.

The biometric characteristic parameter detector is used to detect a real-time biometric characteristic parameter of the user of the wearable apparatus and transmit the detected biometric characteristic parameter to the processor. The biometric characteristic parameter may be physical sign information of the user, and may include various types, such as one or more of a blood parameter, a blood sugar parameter, a blood pressure parameter and a visual parameter. Structure and function of the biometric characteristic parameter detector may be configured based on the type of the biometric characteristic parameter to be detected. Exemplarily, the blood parameter may specifically include one or more of peripheral pulse (PPR), degree of blood oxygen saturation (SpO₂), hematoglobin (Hb), partial pressure of carbon dioxide (PCO₂), partial pressure of oxygen (PO₂), mean arterial pressure (MAP), cardiac output (CO), stroke volume (SV), total carbon dioxide (TCO₂), oxygen content (O₂CT), hematocrit (Hct), red blood cell (RBC), pH value (pH), and blood viscosity (BV). The biometric characteristic parameter detector may be a non-invasive medical examination or detection device by which the physical signs of the user are examined or detected such that non-invasive examination or detection of the physical signs is performed, thereby improving user experience. The non-invasive medical examination device may be implemented by conventional technology and will not be described in detail herein. In an embodiment, the wearable apparatus may include one or more biometric characteristic parameter detectors, which may be disposed at various positions of the wearable apparatus and may detect biometric characteristic parameters at the same position or different positions of the body of the user or detect different types of the biometric characteristic parameters of the user. A plurality of biometric characteristic parameter detectors may be provided to detect the biometric characteristic parameter at the same position of the user or to detect the same type of biometric characteristic parameters, such that the detection may be more accurate and reliable.

The reference biometric characteristic parameter is used to indicate the physical sign(s) of the user is normal and may be set according to medical industry standard. The reference biometric characteristic parameter may be prestored in the processor. The processor may compare the real-time biometric characteristic parameter with the prestored reference biometric characteristic parameter after receiving the real-time biometric characteristic parameter transmitted from the biometric characteristic parameter detector, and determine whether the real-time biometric characteristic parameter is abnormal or not according to the comparison result(s).

A comparison rule for comparing the real-time biometric characteristic parameter with the reference biometric characteristic parameter may be set according to actual situation. For example, if the real-time biometric characteristic parameter exceeds the reference biometric characteristic parameter, the processor determines the real-time biometric characteristic parameter is abnormal and indicates the user may encounter health problem; otherwise, the processor determines the real-time biometric characteristic parameter is normal. Specifically, for example, when a detected blood pressure value is greater than a preset blood pressure threshold, it is determined that the blood pressure of the user is abnormal and the user may encounter a blood pressure potential trouble.

Further, for example, if a difference value between the real-time biometric characteristic parameter and the reference biometric characteristic parameter exceeds a preset difference value, the processor determines the real-time biometric characteristic parameter is abnormal; otherwise, the processor determines the real-time biometric characteristic parameter is normal. In a further example, if a ratio of the real-time biometric characteristic parameter to the reference biometric characteristic parameter exceeds a preset ratio value, the processor determines the real-time biometric characteristic parameter is abnormal; otherwise, the processor determines the real-time biometric characteristic parameter is normal. Other characteristic comparing rules and determining methods may be implemented and any comparing rules and determining method that are suitable to the present disclosure may be implemented.

According to embodiments of the present disclosure, the wearable apparatus has an identity authentication function, biometric characteristic parameter detection function and biometric characteristic parameter determination function, and thus the user may simply and fast implement a biometric characteristic parameter detection and a medical detection or examination by wearing the wearable apparatus, achieving a convenient detection or examination.

In an embodiment, in operation, the processor may further, after receiving the real-time biometric characteristic parameter transmitted from the biometric characteristic parameter detector, control the real-time biometric characteristic parameter to be transmitted by a network to a specified server, such as cloud health data center, which prestores therein standard or reference biometric characteristic parameters and which compares the received real-time biometric characteristic parameter with the standard or reference biometric characteristic parameter and determine whether the real-time biometric characteristic parameter is abnormal or not according to the comparison result. Further, the server may transmit the determination result to the wearable apparatus, and the determination result is outputted by the wearable apparatus to inform the biometric characteristic parameter detection or examination result to the user.

After the identity characteristic extraction device acquires the identity characteristic information, there are various types of subjects for implementing determination of the identity characteristic information. For example, the reference identity characteristic information of an authorized user may be prestored in the identity characteristic extraction device, and, the identity characteristic extraction device may, after acquiring the identity characteristic information of the user of the wearable apparatus, compare the acquired identity characteristic information of the user with the prestored reference identity characteristic information, determine the current user is an authorized user if the comparison is yes, passed or successful, for example, if the acquired identity characteristic information of the user is consistent with or matches the prestored reference identity characteristic information, and transmits the determination result to the processor. Then, the processor controls the biometric characteristic parameter detector to detect the real-time biometric characteristic parameter of the user.

In another example, the identity characteristic extraction device is only used to acquire the identity characteristic information of the user of the wearable apparatus and transmit the acquired identity characteristic information of the user to the processor. The processor prestores reference identity characteristic information of an authorized user and is configured to compare the acquired identity characteristic information with the reference identity characteristic information after receiving the acquired identity characteristic information transmitted from the identity characteristic extraction device. If the comparison is yes, passed or successful, for example, if the acquired identity characteristic information of the user is consistent with or matches the prestored reference identity characteristic information, the processor controls the biometric characteristic parameter detector to detect the real-time biometric characteristic parameter of the user.

In the wearable apparatus provided in embodiments of the present disclosure, the processor may be further configured to, when it determines the real-time biometric characteristic parameter is abnormal, analyze the real-time biometric characteristic parameter and output the health information. The health information may include various types, for example, at least one of a potential disease that the user may encounter and recommendations for health improvement.

In addition, the wearable apparatus may further include a display device electrically connected with the processor. The display device may be configured to display the health information outputted by the processor. The display device may include a display. With the display device, the wearable apparatus may achieve display function of the health information such that the user may know the detected health information by viewing content(s) displayed by the display device, which further enriches the functions of the wearable apparatus and improves user experience.

The wearable apparatus may further include an audio player, which is electrically connected with the processor and is configured to play in a form of voice the health information outputted by the processor. The user may get his/her health information by listening the audio information played by the wearable apparatus. With the audio player, the wearable apparatus is provided with a function of playing in a form of voice the health information such that the user may get his/hers health information by listening the voice information played by the wearable apparatus, and thus the he wearable apparatus is further improved in terms of functions, improving user experience.

Also, the wearable apparatus provided in the embodiments of the present disclosure may further include a touch device, which is electrically connected with the processor and configured to receive the identity authentication information entered by the user when comparison of the identity characteristic information with the reference identity characteristic information is not passed, for example, when the identity characteristic information is not consistent with or does not match the reference identity characteristic information. In an embodiment, the processor may be configured to control the biometric characteristic parameter detector to detect the real-time biometric characteristic parameter of the user according to the identity authentication information entered by the user. Exemplarily, the touch device may be separately disposed on the body of the wearable apparatus, or disposed in the display device, or integrally formed with the display device, for example, the display device may be a touch display device.

In some examples, the reference identity characteristic information may be identity characteristic information of a user authorized by the wearable apparatus. The reference identity authentication information may be prestored by the user authorized by the wearable apparatus, and may include various forms, such as one of or a combination of two or more of digital, character, wording, graph and track.

According to embodiments of the present disclosure, after acquiring the identity characteristic information by the identity characteristic extraction device, if the identity characteristic extraction device is used to compare the acquired identity characteristic information with the preset reference identity characteristic information and if the comparison is not passed, for example, if the identity characteristic information is not consistent with to or does not match the preset reference identity characteristic information, the identity characteristic extraction device transmits the comparison result to the processor; then, the processor will transmit an enable instruction to enable the touch device. If the processor is used to compare the acquired identity characteristic information with the preset reference identity characteristic information, the processor transmits the enable instruction to enable the touch device if the comparison is not passed.

The wearable apparatus may transmit an indication to the user that the authentication of the identity characteristic information is not passed or is failed. Exemplarily, if the wearable apparatus includes a display screen, the above indication may be displayed on the display screen; or, the wearable apparatus may output voice indication to inform the user that the authentication of the identity characteristic information is not passed or is failed. Other indication manners may be applicable. Subsequently, the touch device may receive identity authentication information entered by the user and transmit the identity authentication information to the processor. The processor, after receiving the identity authentication information entered by the user and transmitted from the touch device, compares the received identity authentication information with prestored reference identity authentication information, and controls the biometric characteristic parameter detector to detect the real-time biometric characteristic parameter of the user if the comparison is yes or passed, for example, if the received identity authentication information is consistent with to or matches the prestored reference identity authentication information.

The touch device may be a panel having a touch sensitive function. Further, the touch device may further have a displaying function. When the touch device is a touch display panel, data/information outputted or received by the wearable apparatus may be displayed on the touch display panel. For example, the above indication that the authentication of the identity characteristic information is not passed or failed, the health information, the detected biometric characteristic parameter, the detected identity characteristic information of the user and the like may be displayed on the touch device for viewing by the user.

Provision of the touch device adds a way of authenticating the identity of the user for the wearable apparatus. For example, if authentication of the identity characteristic information acquired by identity characteristic extraction device is failed, the wearable apparatus may control the touch device to perform the identity authentication again, which enriches function of the wearable apparatus.

The wearable apparatus according to embodiments of the present disclosure may further include a distance detection device, which is electrically connected with the processor and configured to detect a distance between the wearable apparatus and at least one electronic apparatus. The processor may be further configured to determine whether or not the wearable apparatus mates with the at least one electronic apparatus according to the detected distance, and to control the at least one electronic apparatus to perform at least one operation when the wearable apparatus successfully mates with the at least one electronic apparatus.

The above electronic apparatus may be of various types, such as a room door lock, a car lock, a household electrical appliance, a vehicle, a window, a mobile phone, a computer or the like. A distance threshold condition between the wearable apparatus and the electronic apparatus may be preset in the processor, and the processor determines that the wearable apparatus successfully mates with the electronic apparatus if the detected distance is less than the distance threshold when determining whether the detected distance meets the distance threshold condition. Then, the processor may control the electronic apparatus to perform a corresponding operation. The operation of the electronic apparatus controlled by the processor may include various types, such as on or off of the electronic apparatus, locking or unlocking of the electronic apparatus, opening or closing of a vehicle door, or the like. For example, if the wearable apparatus is a glove and the electronic apparatus is a room door lock, it is determined that the glove successfully mates with the door lock if a distance between the glove and the door lock is determined to be less than a preset distance threshold, and then the door lock is controlled to be opened automatically.

With the distance detecting device, the wearable apparatus is provided with a function of controlling the electronic apparatus to perform a corresponding operation such that the user may control the electronic apparatus just by wearing the wearable apparatus, thereby saving user's associated operations on the electronic apparatus, enhancing functions of the wearable apparatus and improving the user experience.

The wearable apparatus provided in embodiments of the present disclosure may include the distance detection device and a communication control device, which are electrically connected with the processor. The distance detection device is configured to detect the distance between the wearable apparatus and the electronic apparatus; the communication control device is configured to transmit the identity identification information to the electronic apparatus when the detected distance meets the distance threshold condition, such that the electronic apparatus verifies the identity identification information and controls its working state according to the verification result. The identity identification information may be of various types, such as the identity characteristic information acquired by the identity characteristic extraction device in the wearable apparatus when the user wears the wearable apparatus.

The electronic apparatus may prestore therein the distance threshold condition and the identity identification information of the authorized user. The distance threshold condition may be set according to actual situation. For example, the distance threshold condition is met if the detected distance is less than the preset distance. The distance detecting device may include a distance sensor or other type of distance detection device.

When the user uses the wearable apparatus, the distance detection device of the wearable apparatus will detect the distance between the wearable apparatus and the electronic apparatus. When the detected distance between the wearable apparatus and the electronic apparatus meets the distance threshold condition, the communication control device transmits the current identity characteristic information of the user acquired by the identity characteristic extraction device to the electronic apparatus. The electronic apparatus compares, after receiving the current identity characteristic information of the user transmitted from the wearable apparatus, the current identity characteristic information of the user with the prestored identity characteristic information of the authorizated user; if it is compared that the current identity characteristic information of the user matches with the prestored identity characteristic information of the authorized user, it is determined that authentication of the current identity characteristic information of the user is successful and the current user is determined to be an authorized user; then the electronic apparatus starts to work.

In another embodiment, the electronic apparatus is an air conditioner. If the distance between the wearable apparatus and the electronic apparatus meets the distance threshold condition, the user may directly activate the air conditioner to work by wearing the wearable apparatus at a short distance from the electronic apparatus. In an embodiment, the electronic apparatus is a door lock. If the distance between the wearable apparatus and the door lock meets the distance threshold condition, specifically for example, the wearable apparatus, such as, a smart glove, is placed on the door lock, the user may directly unlock the door lock by wearing the smart glove without using any key or door card, achieving convenient operation.

The wearable apparatus according to embodiments of the present disclosure may further include an air detection device, which is electrically connected with the processor and may be configured to detect air quality in an environment where the wearable apparatus is located. The processor may further be configured to feed back the air quality to the user.

The user may transmit an instruction of initiating an air detecting operation to the processor by a preset operation and the processor may transmit an initiation instruction to the air detection device so as to control the air detection device to start to work. The air detection device may transmit the detected air quality to the processor, so that the processor may feed back the detected air quality to the user.

When the wearable apparatus is provided with a display screen, the processor is electrically connected with the display screen and the processor may display the air quality fed back by the air detection device on the display screen for viewing by the user. When the wearable apparatus is provided with a signal light, the processor is electrically connected with the signal light and then the processor may indicate a level of the air quality by controlling a displaying color of the signal light. When the wearable apparatus is provided with an audio output device, the processor is electrically connected with the audio output device and then the processor may output the air quality in an audio or voice form for listening by the user, or the air quality may be presented in other suitable form.

Provision of the air detection device makes the wearable apparatus have a function of detecting the air quality, and the user may know the environment air quality therearound by using the wearable apparatus, thereby further enhancing functions of the wearable apparatus.

In an embodiment of the present disclosure, the wearable apparatus may further include or be provided with a database, and parameters and conditions used in the above operations, such as, the reference biometric characteristic parameter, the reference identity characteristic information, and the distance threshold condition, may be stored in the database such that desired parameter(s) or condition(s) may be read or retrieved from the database and used by the processor. The database may be programmed in the processor or may be stored in a separate data memory which is independent of the processor and may be connected with the processor as required.

The wearable apparatus according to embodiments of the present disclosure may be of various types, such as a glove, glasses, etc. A glove may use detected fingerprint information of the user as the identity characteristic information and perform detection or examination of characteristics information such as blood, blood pressure, blood sugar, etc. of the user. The glove may use detected pupil information of the user as the identity characteristic information and perform detection or examination of characteristics information such as vision of the user. The forms of the wearable apparatus are only described herein for examples and will not be limited herein, and any of suitable forms of the wearable apparatus may be implemented.

When the wearable apparatus is a glove, the identity characteristic extraction device may be disposed at a fingertip portion of the glove and configured to acquire fingerprint information of the user when the user wears the wearable apparatus. Based on the above configuration of the identity characteristic extraction device, the wearable apparatus may having a fingerprint detection function and may make determination of the fingerprint information so as to achieve authentication of the identity characteristic information of the user.

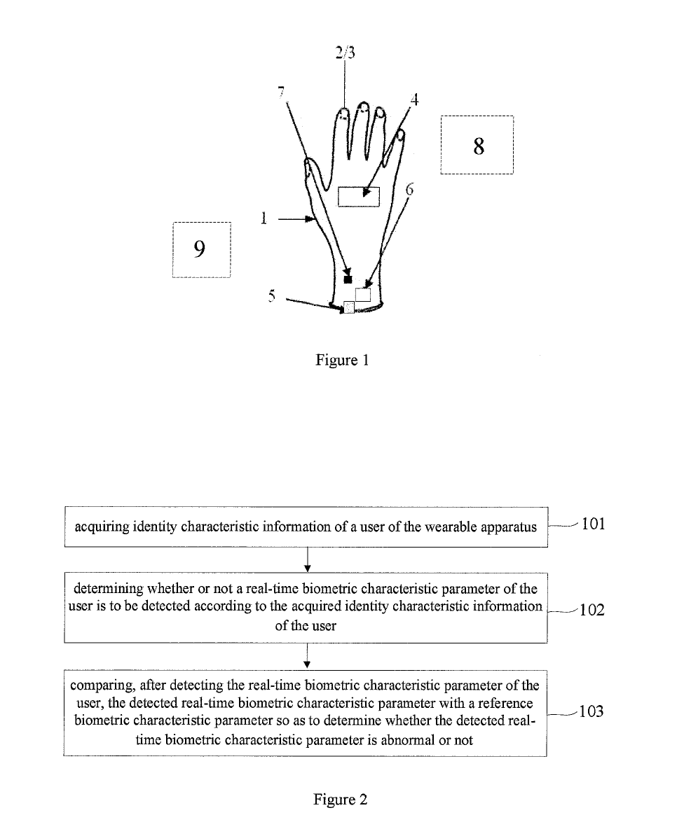

FIG. 1 is a structural schematic view of a smart glove according to an embodiment of the present disclosure. As shown in FIG. 1, the smart glove includes a glove body 1, a fingerprint identification sensor 2, a non-invasive blood test device 3 and a processor 6. In an embodiment, the fingerprint identification sensor 2 and the non-invasive blood test device 3 are respectively disposed at positions of the fingertip of the glove body 1. Although it is shown in FIG. 1 that the fingerprint identification sensor 2 and the non-invasive blood test device 3 are positioned at a same fingertip portion of the glove body 1, the fingerprint identification sensor 2 and the non-invasive blood test device 3 may be positioned at different fingertip portions of or at different positions of a same fingertip portion of the glove body 1 in other embodiments. In some embodiments, the processor 6 includes or is provided with a database which stores therein various threshold(s), preset condition(s) and the like that is/are to be used during usage of the smart glove. Alternately, the database may be provided in a server 8 in communication with the wearable apparatus. In some embodiments, the wearable apparatus, such as the smart glove, may further include a display device 4 and a distance detection device 5 configured to detect a distance between the wearable apparatus and an electronic apparatus 9. As shown in the Figure, the display device 4 may be located at a back side of the globe body 1, facilitating viewing by the user. In another embodiment, the wearable apparatus, such as the smart glove, may further include a communication control device 7, such as a signal transmitter and/or receiver, configured for enabling communication between the wearable apparatus and the server 8. The communication control device 7 is in communication with or is electrically connected with the processor 6. Structure, function and connection relationship of the above components of the smart glove are similar to those described in the above embodiments and are not repeatedly described herein.

Based on the structure and function of the smart glove shown in FIG. 1, the smart glove may function to achieve fingerprint detection, fingerprint identification, medical examination, and medical data analysis and output. In addition, the smart glove may cooperate with the electronic apparatus such that the user may, by wearing the smart glove, control the electronic apparatus to perform an operation, for example, control the electronic apparatus to start working, achieving convenient control of the electronic apparatus 9 by the user.

Embodiments of the present disclosure further provide a method of controlling a wearable apparatus. FIG. 2 is a flow chart of a method of controlling a wearable apparatus according to an embodiment of the present disclosure. The method may be applied to the wearable apparatus provided by the embodiments of the present disclosure. The method mainly includes the following steps:

Step 101: acquiring identity characteristic information of a user of the wearable apparatus;

Step 102: determining whether or not a real-time biometric characteristic parameter of the user is to be detected according to the acquired identity characteristic information of the user; and

Step 103: comparing the detected real-time biometric characteristic parameter with a reference biometric characteristic parameter after detecting the real-time biometric characteristic parameter of the user, so as to determine whether the real-time biometric characteristic parameter is abnormal or not.

According to the embodiments of the present disclosure, the wearable apparatus has functions including identity authentication, biometric characteristic parameter detection and biometric characteristic parameter determination such that the user may simply and fast perform biometric characteristic parameter detection or examination just by wearing the wearable apparatus, achieving convenient detection.

Further, the step of determining whether or not a real-time biometric characteristic parameter of the user is to be detected according to the identity characteristic information of the user may include:

comparing the identity characteristic information of the user with the reference identity characteristic information, and, detecting the real-time biometric characteristic parameter of the user if the comparison is passed, for example, if the identity characteristic information of the user is consistent with or matches the reference identity characteristic information.

In an embodiment, the method may further include:

analyzing the real-time biometric characteristic parameter and outputting health information when determining the real-time biometric characteristic parameter is abnormal.

Further, the health information may comprise at least one of a potential disease of the user and recommendation for health improvement for the user.

Also, the identity characteristic information may include fingerprint information of the user, and the real-time biometric characteristic parameter includes at least one of a blood parameter, a blood sugar parameter and a blood pressure parameter of the user.

In another embodiment, the method may further include: receiving, if the comparison is not passed or is failed, for example, if the identity characteristic information is not consistent with or does not match the reference identity characteristic information, identity authentication information entered by the user to perform identity authentication of the user so as to determine whether or not the real-time biometric characteristic parameter of the user is to be detected according to the identity authentication result.

In other embodiments, the method may further include steps of:

detecting a distance between the wearable apparatus and at least one electronic apparatus;

determining whether or not the wearable apparatus mates with the at least one electronic apparatus according to the detected distance; and

controlling the at least one electronic apparatus to perform at least one operation when the wearable apparatus mates with the at least one electronic apparatus.

Further, the step of determining whether or not the wearable apparatus mates the at least one electronic apparatus according to the detected distance may include:

comparing the detected distance with a distance threshold, and controlling the wearable apparatus to mate with the at least one electronic apparatus when the detected distance is less than the distance threshold.

In the embodiments of the present disclosure, the wearable apparatus includes a body and an identity characteristic extraction device, a biometric characteristic parameter detector and a processor disposed onto the body; the processor are respectively electrically connected with the identity characteristic extraction device and the biometric characteristic parameter detector such that the wearable apparatus has an identity authentication function, a biometric characteristic parameter detection or examination function and a biometric characteristic parameter determination function. With this configuration, the user may simply and fast perform biometric characteristic parameter detection or examination by wearing the wearable apparatus, achieving convenient detection or examination process.

In the embodiments of the present disclosure, the above functional modules/devices may be respectively implemented in the wearable apparatus in a hardware, software and/or firmware, and for example, may be implemented solely or collectively by one or more logical calculation and processing circuit. The logical calculation and processing circuit may be a processor, such as a central processing unit (CPU), or may be an application specific integrated circuit (ASIC), or may be a digital signal processor (DSP), or may be a field programmable gate array (FPGA), or may be a single-chip microcomputer (MCU), etc.

In this context, unless specifically stated otherwise, or as is apparent from the discussion, terms such as “processing” or “computing” or “calculating” or “determining” or “displaying”, “comparing” or the like, refer to the action and processes of a computer system, or similar electronic computing device, that manipulates and transforms data represented as physical, electronic quantities within the computer system's registers and memories into other data similarly represented as physical quantities within the computer system memories or registers or other such information storage, transmission or display devices.

In the above description, exemplary embodiments have been described with reference to acts and symbolic representations of operations (e.g., in the form of flowcharts) that may be implemented as program modules or functional processes including routines, programs, objects, components, data structures, etc., that perform particular tasks or implement particular abstract data types and may be implemented using existing hardware.

It will be understood by those skilled in the art that the present disclosure includes devices for implementing one or more of methods, steps, operations or functions of modules in the present application. These devices may be specially designed and manufactured for desired purposes, or may include known devices in general-purpose computers. These devices have computer programs stored therein which are selectively activable or reconstructable. Such computer programs may be stored in a device (for example, computer) readable medium or in any kind of medium adapted to store electronic instructions therein and to be coupled with a bus, the computer readable medium includes but is not limited any type of disk (including floppy disk, hard disk, compact disc, CD-ROM and magneto-optical disk), ROM (Read-Only Memory), RAM (Random Access Memory), EPROM (Erasable Programmable Read-Only Memory), EEPROM (Electrically Erasable Programmable Read-Only Memory), flash, magnetic card or optical card. That is, the readable medium include any medium for storing or transmitting information therein in readable form devices (for example, computer).

It will be appreciated that the above embodiments are only exemplary embodiments for illuminating inventive concepts of the present disclosure, and are not intended to limit the present disclosure, the scope of which is defined in the claims and their equivalents. Various changes or modifications may be made by those skilled in the art without departing from the principle and spirit of the present disclosure and shall fall within the scope of the present disclosure.

What is claimed is:

1. A wearable apparatus, comprising a body and an identity characteristic extraction device, a biometric characteristic parameter detector and a processor that are disposed onto the body; wherein
the identity characteristic extraction device is electrically connected with the processor and is configured to acquire identity characteristic information of a user of the wearable apparatus;
the biometric characteristic parameter detector is electrically connected with the processor and is configured to detect a real-time biometric characteristic parameter of the user; and
the processor is configured to, according to the acquired identity characteristic information, control the biometric characteristic parameter detector to detect the real-time biometric characteristic parameter of the user and compare the detected real-time biometric characteristic parameter with a reference biometric characteristic parameter so as to determine whether the detected real-time biometric characteristic parameter is abnormal or not.

2. The wearable apparatus according to claim 1, wherein the processor is configured to compare the acquired identity characteristic information of the user with the reference identity characteristic information, and, in response to matching between the acquired identity characteristic information of the user and the reference identity characteristic information, control the biometric characteristic parameter detector to detect the real-time biometric characteristic parameter of the user.

3. The wearable apparatus according to claim 1, wherein the processor is further configured to, in response to determining the detected real-time biometric characteristic parameter is abnormal, analyze the detected real-time biometric characteristic parameter and output health information.

4. The wearable apparatus according to claim 3, wherein the health information comprises at least one of a potential disease of the user and recommendation for health improvement for the user.

5. The wearable apparatus according to claim 1, wherein the identity characteristic information includes fingerprint information of the user, and the real-time biometric characteristic parameter includes at least one of a blood parameter, a blood sugar parameter and a blood pressure parameter of the user.

6. The wearable apparatus according to claim 3, wherein the wearable apparatus further comprises a display device electrically connected with the processor and configured to display the health information.

7. The wearable apparatus according to claim 2, further comprising a touch device electrically connected with the processor and configured to, in response to mismatching between the acquired identity characteristic information and the reference identity characteristic information, receive identity authentication information entered by the user, and
wherein the processor is further configured to, according to the received identity authentication information, control the biometric characteristic parameter detector to detect the real-time biometric characteristic parameter of the user.

8. The wearable apparatus according to claim 1, further comprising a distance detection device electrically connected with the processor and configured to detect a distance between the wearable apparatus and at least one electronic apparatus; and
wherein the processor is further configured to, according to the detected distance, determine whether or not the wearable apparatus mates with the at least one electronic apparatus and control the at least one electronic apparatus to perform at least one operation in response to success mating of the wearable apparatus with the at least one electronic apparatus.

9. The wearable apparatus according to claim 1, further comprising an air detection device electrically connected with the processor and configured to detect air quality in an environment where the wearable apparatus is located, and
wherein the processor is further configured to feed back the air quality to the user.

10. The wearable apparatus according to claim 1, wherein the wearable apparatus is a glove, and the identity characteristic extraction device is disposed at a fingertip portion of the glove and configured to acquire the user's fingerprint information in a state where the user wears the glove.

11. The wearable apparatus according to claim 1, wherein the biometric characteristic parameter detector comprises a non-invasive medical examination device.

12. The wearable apparatus according to claim 3, further comprising an audio player electrically connected with the processor and configured to play in a form of voice the health information outputted by the processor.

13. A method of controlling a wearable apparatus, the method comprising:
acquiring identity characteristic information of a user of the wearable apparatus;
determining whether or not a real-time biometric characteristic parameter of the user is to be detected according to the acquired identity characteristic information of the user; and
comparing, after detecting the real-time biometric characteristic parameter of the user, the detected real-time biometric characteristic parameter with a reference biometric characteristic parameter so as to determine whether the detected real-time biometric characteristic parameter is abnormal or not.

14. The method according to claim 13, wherein the determining whether or not a real-time biometric characteristic parameter of the user is to be detected according to the identity characteristic information of the user comprises:
comparing the acquired identity characteristic information of the user with the reference identity characteristic information, and, in response to matching between the acquired identity characteristic information of the user and the reference identity characteristic information, detecting the real-time biometric characteristic parameter of the user.

15. The method according to claim 13, wherein the method further comprises:
analyzing the detected real-time biometric characteristic parameter and outputting health information when determining the detected real-time biometric characteristic parameter is abnormal.

16. The method according to claim 15, wherein the health information comprises at least one of a potential disease of the user and recommendation for health improvement for the user.

17. The method according to claim 13, wherein the identity characteristic information includes fingerprint information of the user, and the real-time biometric characteristic parameter includes at least one of a blood parameter, a blood sugar parameter and a blood pressure parameter.

18. The method according to claim 14, wherein the method further comprises:
receiving identity authentication information entered by the user to perform identity authentication for the user when the identity characteristic information does not match the reference identity characteristic information, and, according to identity authentication result, determining whether or not the real-time biometric characteristic parameter of the user is to be detected.

19. The method according to claim 13, wherein the method further comprises:
detecting a distance between the wearable apparatus and at least one electronic apparatus;
determining whether or not the wearable apparatus mates with the at least one electronic apparatus according to the detected distance; and
controlling the at least one electronic apparatus to perform at least one operation when the wearable apparatus successfully mates with the at least one electronic apparatus.

20. The method according to claim 19, wherein the determining whether or not the wearable apparatus mates with the at least one electronic apparatus according to the detected distance comprises:
comparing the detected distance with a distance threshold, and controlling the wearable apparatus to mate with the at least one electronic apparatus when the detected distance is less than the distance threshold.
